(19) United States
(12) Patent Application Publication
(10) Pub. No.: US 2019/0122770 A1
(43) Pub. Date: Apr. 25, 2019

(54) Lightweight Clinical Pregnancy Preterm Birth Predictive System and Method

(71) Applicant: Parkland Center for Clinical Innovation, Dallas, TX (US)

(72) Inventors: Yolande Mfondoum Pengetnze, Flower Mound, TX (US); Weiwei Ouyang, Minnetonka, MN (US); Steve Miff, Dallas, TX (US); Albert Karam, Dallas, TX (US); Xiao Wang, Dallas, TX (US)

(21) Appl. No.: 16/228,732

(22) Filed: Dec. 20, 2018

Related U.S. Application Data
(63) Continuation-in-part of application No. 14/514,164, filed on Oct. 14, 2014.
(60) Provisional application No. 61/891,054, filed on Oct. 15, 2013.

(51) Int. Cl. G16H 50/30; G06N 20/00; G06N 5/04; G16H 50/20

(52) U.S. Cl. CPC G16H 50/30; G16H 50/20; G06N 5/04; G06N 20/00

(57) ABSTRACT

A lightweight clinical pregnancy preterm birth predictive system includes a data store configured to receive and store patient data consisting of only health insurance claim data associated with a plurality of patients, a predictive model including a plurality of weighted risk variables and risk thresholds, a risk logic module configured to identify a pool of pregnant patients and to apply the predictive model to the patient data of the pool of pregnant patients to determine a risk score for each pregnant patient to identify at least one patient who is at risk for preterm birth, and a data presentation module operable to present notification and information to an intervention coordination team about the identified at least one high-risk patient.

### RELATED APPLICATION

This patent application is a continuation-in-part of U.S. application Ser. No. 14/514,164 filed on Oct. 14, 2014, which claims the benefit of U.S. Provisional Application No. 61/891,054 filed on Oct. 15, 2013, all of which is incorporated herein by reference. This application is also related to the following patents, all of which are incorporated herein by reference: U.S. patent application Ser. No. 16/194,277 filed Nov. 16, 2018, entitled “System and Method for a Payment Exchange Based on an Enhanced Patient Care Plan”; U.S. application Ser. No. 13/613,980 filed Sep. 13, 2012, now U.S. Pat. No. 9,536,052 entitled “Clinical Predictive and Monitoring System and Method”; and U.S. application Ser. No. 14/018,514 filed Sep. 5, 2013, now U.S. Pat. No. 9,147,041 entitled “Clinical Dashboard User Interface System and Method.”

### FIELD

The present disclosure relates to a lightweight clinical pregnancy preterm birth predictive system and method.

### BACKGROUND

Preterm birth is when a baby is born too early, commonly defined as birth before 37 weeks of gestation. In 2016, preterm birth affected about 1 of every 10 infants born in the United States. On the decline for many years, the rate of preterm birth in the United States began to increase in 2015. Worldwide, an estimated 15 million babies are born too early every year, and approximately 1.1 million infants die each year due to complications of preterm birth. Globally, prematurity is the leading cause of death and long-term disability in children under the age of 5 years. In almost all countries with reliable data, preterm birth rates are increasing.

Preterm birth not only causes needless loss of lives, it is also a significant monetary drain on society. A baby delivered at term costs approximately $5,000.00, while a premature baby costs the healthcare system approximately $50,000.00, at least a ten-fold increase. Many survivors face a lifetime of physical, neurological, and learning disabilities, resulting in great cost to family and society. An estimate for the cost associated with premature birth in the United States is approximately $31.5 billion per year (source: 2006 Institute of Medicine published data adjusted to rate of inflation).

Preventing deaths and complications from preterm birth starts with a healthy pregnancy. Quality maternal care before and during pregnancies would go a long way toward ensuring a full-term birth. Key interventions to help prevent preterm birth include counselling on healthy diet and optimal nutrition, and tobacco and substance use; fetal measurements including use of ultrasound to help determine gestational age and detect multiple pregnancies; and a minimum of eight contacts with health professionals throughout pregnancy to identify and manage other risk factors, such as infections.

### BRIEF DESCRIPTION OF THE DRAWINGS

FIG. 1 is a simplified block diagram of an exemplary embodiment of a clinical predictive and monitoring system and method according to the present disclosure;

FIG. 2 is a timeline diagram of an exemplary embodiment of a clinical predictive and monitoring system and method according to the present disclosure;

FIG. 3 is a simplified logical block diagram of an exemplary embodiment of a clinical predictive and monitoring system and method according to the present disclosure;

FIG. 4 is a simplified flowchart of an exemplary embodiment of a clinical predictive and monitoring method according to the present disclosure; and

FIG. 5 is a simplified flowchart/block diagram of an exemplary embodiment of a clinical predictive and monitoring method according to the present disclosure.

### DETAILED DESCRIPTION

Surprisingly, about fifty percent of women who go on to deliver early do not show any symptoms for preterm birth. The best indication that a woman may deliver early is if she's had a previous premature birth. However, about forty percent of women who deliver prematurely are first-time mothers, making the identification of preterm birth risk extremely challenging.

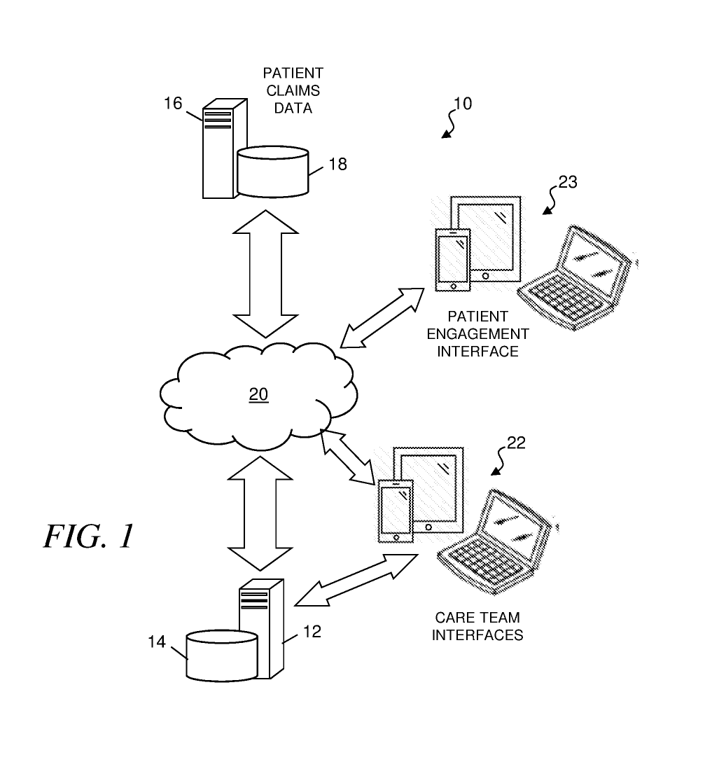

FIG. 1 is a simplified block diagram of an exemplary embodiment of a lightweight clinical pregnancy preterm birth predictive system and method 10 using a predictive engine to analyze only health insurance claims data according to the teachings of the present disclosure. The system and method 10 include a computer system 12 adapted to receive and analyze patient data such as the patient's insurance (e.g., Medicaid) claims data relating to patients or individuals. The system and method 10 are termed “lightweight” because only insurance claims data are analyzed. In an exemplary implementation, one or more social determinant of health factors that are derived from claims data or non-claims data may additionally be used. For example, the zip code level median income is derived from Census data. The claims and supplemental data are used to identify patients who, if pregnant, would be at risk of preterm delivery/birth so that they may receive more targeted intervention, treatment, and care that would help them to carry their babies to full-term. It should be noted that the computer system 12 may comprise one or more local or remote computer servers operable to transmit data and communicate via wired and wireless communication links and computer networks. The system 10 also includes one or more database 14 that is used to store the patient data. The patient claims data may originate from an insurance computer system 16 and database 18 transmitted over a global computer network 20. The system 10 also includes interfaces 22 and 23 that permit authorized access by the healthcare team and the patient respectively via a variety of computing devices (e.g., mobile devices, tablet computers, laptop computers, desktop computers, servers, etc.). The data may be presented and displayed to the healthcare team/patient in the form of web pages, web-based message, text files, video messages, multimedia messages, text messages, e-mail messages, mobile applications, and in a variety of suitable formats.

As shown in FIG. 1, the system 10 may receive the claims data streamed real-time, or from historic or batched data from one or more data sources. The real-time and stored data may be in a wide variety of formats according to a variety of protocols, including CCD, XDS, HL7, SSO, HTTPS, EDI, CSV, etc. The data may be encrypted or otherwise secured in a suitable manner. The claims data may be pulled (polled) by the system 10 from the various data sources or the data may be pushed to the system 10 by the data sources. Alternatively or in addition, the data may be received in batch processing according to a predetermined schedule or on-demand. Alternatively or in addition, the data may be stored in a data center in the cloud.

The computer system 12 may comprise a number of computing devices, including servers, that may be located locally or in a cloud computing farm. The data paths between the computer system 12 and 16 is preferably encrypted or otherwise protected with security measures or transport protocols now known or later developed. Further the claims data may also be anonymized while in transit to conform with HIPAA and/or other regulations concerning medical data privacy.

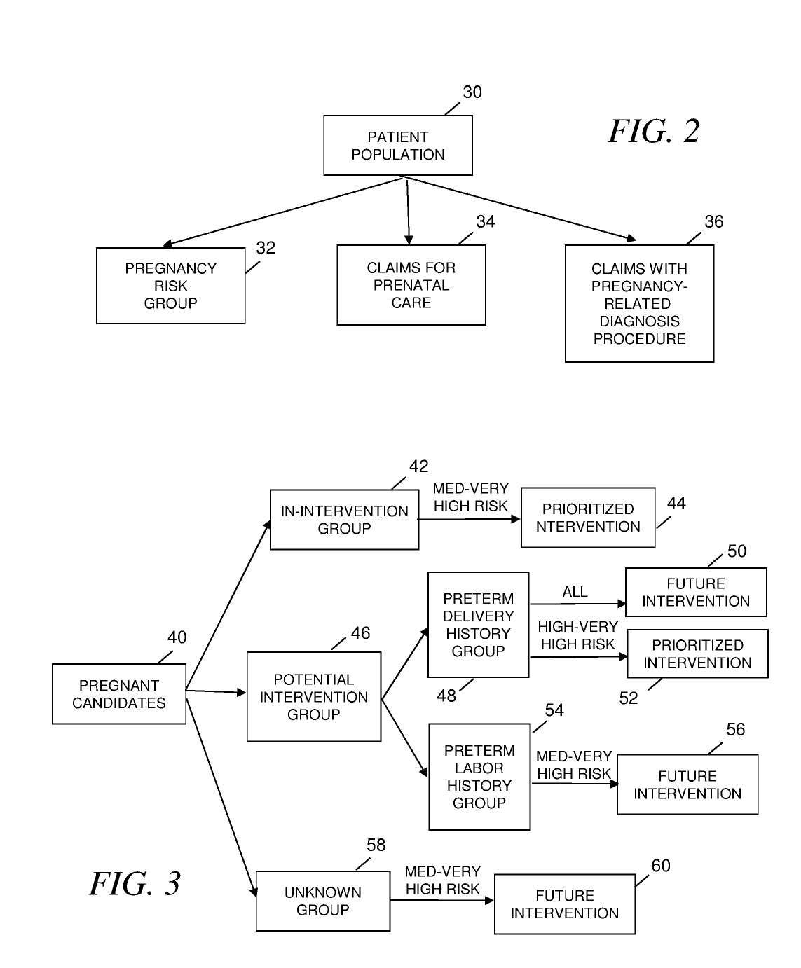

FIG. 2 is a block diagram illustrating the process by which pregnant women are identified by analyzing only insurance claims data according to the teachings of the present disclosure. Block 30 represents the entire patient population. The insurance claims data of the entire patient population are analyzed to identify women who are pregnant according to three criteria: claims data that identify a particular patient to be a Medicaid Type Programs 40 (TP 40) 32; claims data indicating that the patient is receiving prenatal care services 34; and claims data indicating that the patient is receiving pregnancy-related diagnosis or procedures 36. Any patient who meets one or more of the criteria 32-36 is automatically included in the pregnant cohort for risk prediction, reporting, and intervention.

FIG. 3 is a block diagram illustrating the process by which pregnant women are further identified as requiring prioritized or future intervention from analyzing only insurance claims data according to the teachings of the present disclosure. From the pregnant women patient cohort identified by the process shown in FIG. 2, those women whose claims data that include certain indicators are identified as the in-intervention group 42 requiring prioritized intervention. The in-intervention group 42 are women whose claims data indicate that, for example, they are receiving hydroxy progesterone caproate (17-OH Progesterone) therapy or cervical cerclage, which are known treatments for preventing preterm birth. The claims data of these women in the in-intervention group 42 are analyzed by the computer system and method 10, and those women who are evaluated by the predictive engine to be at medium, high, and very high risk for preterm birth are flagged to receive immediate prioritized and targeted intervention 44. A second group 46 of pregnant women are identified from analyzing their claims data that indicate these women have had a history of past preterm labor and/or preterm delivery. These women are classified as the potential intervention group 46. The women in this potential intervention group 46 are further divided into a preterm delivery history group 48 and a preterm labor history group 54. All of the women in the preterm delivery history group 48 will be continually monitored and evaluated for future change in risk status that would warrant more prioritized intervention throughout their pregnancy 50. However, those women in this group 48 that have been evaluated by the predictive engine to be at high to very high risk for preterm birth, will receive prioritized and targeted intervention 52. Women who have a history of preterm labor 54 and evaluated by the predictive engine to be at medium to very high risk for preterm birth, will be continually monitored and evaluated for future change in risk status that would warrant more prioritized intervention throughout their pregnancy 56. A third group of pregnant women, labeled as the unknown group 58, are women who do not have a history of preterm labor or birth, and are not known to be receiving hydroxy progesterone caproate (17-OH Progesterone) therapy or cervical cerclage treatment. The women in the unknown group 58 are also evaluated by the predictive engine, and those women who have been identified as having medium to very high risk from this group will be continually monitored and evaluated for future change in risk status that would warrant more prioritized intervention throughout their pregnancy 60.

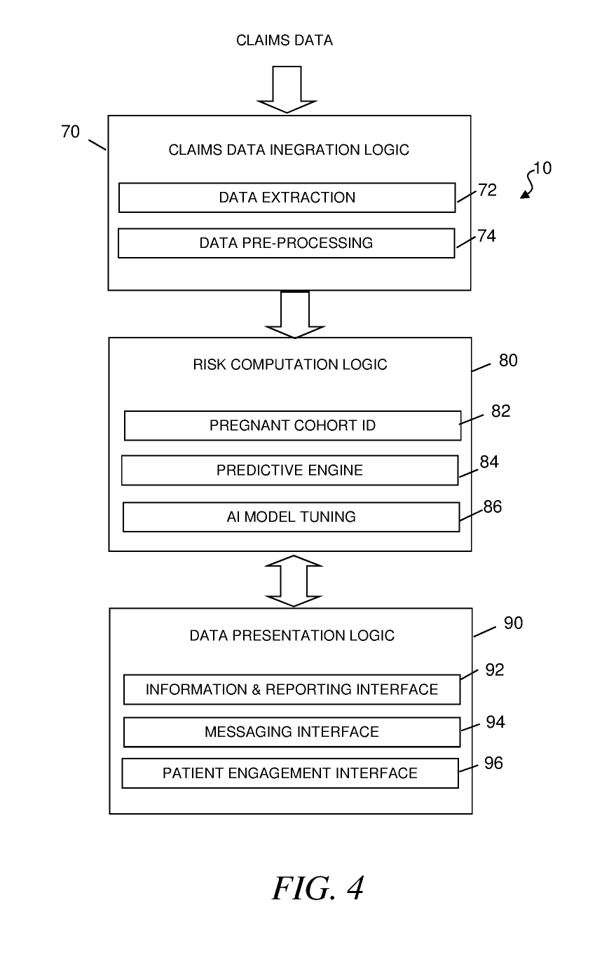

FIG. 4 is a simplified logical block diagram of an exemplary embodiment of a lightweight clinical pregnancy preterm birth predictive system and method 10 according to the present disclosure. The lightweight clinical pregnancy preterm birth predictive system and method 10 includes a claims data integration logic module 70 that further includes a data extraction process 72. The data extraction process 72 extracts the insurance claims data from data records received from one or more data sources in real-time or in batches either directly or through a global network or the Internet. The data extraction process 72 basically pre-processes the received data records to extract relevant claims data that can be analyzed by the predictive engine. The claims data includes data related to the patient's medical claims, pharmacy claims, behavioral and psychiatric claims, eligibility for coverage, insurance membership, and healthcare provider. The claims record may include data on or related to type of claim (inpatient, long term care, prescription drug, etc.), type of service, beginning and end date of service, place of service, type of service, procedure code(s), diagnosis code(s), provider ID(s), prescribing physician ID, prescription, prescription fill date, etc. The eligibility data record may include data on or related to identification number(s), date of birth, gender, race/ethnicity, county, zip code, plan type, basis of eligibility, eligibility group, days of eligibility, income level, etc. In a preferred embodiment of the system 10, a zip code-level income ratio generated from Census information is also received as input data. This ratio provides additional insight to the patient's income level, and may be calculated by the mean income for a particular patient's zip code divided by the lowest mean income for a certain region, such as Dallas-Fort Worth, for example.

The claims data integration logic module 70 further includes a data pre-processing logic module 74 that performs data scrubbing and formatting tasks to remove extraneous data bits, and distill the data down to the specific information in the correct format that are then used in data analytics in the predictive engine 84 described below.

The data integration logic module 70 then passes the pre-processed claims data to a risk computation logic module 80. The risk computation logic module 80 is operable to calculate a risk score for preterm birth for each pregnant patient and identifying those patients who should receive targeted intervention and care. The pregnant cohort identification logic module 82 is configured to identify those patients who are pregnant from just the insurance claims data. As described above, the pregnant cohort identification logic module 82 may identify pregnant women according to three criteria: claims data that identify a particular patient to be a Medicaid Type Programs 40 (TP 40); claims data indicating that the patient is receiving prenatal care services; and claims data indicating that the patient is receiving pregnancy-related diagnosis or procedure. The claims data of patients that are identified as pregnant are then provided to a predictive engine module 84. The predictive engine 84 then analyzes the claims and pharmacy data and computes a risk score for each pregnant patient. In general, the predictive engine 84 may take into account a set of risk factors or variables that may be compared to predetermined thresholds and/or value ranges, where the variables can be weighted. The predictive engine 84 specifies how to categorize and weight each variable or risk factor, and how to calculate the predicted probability of preterm risk. Some of the variables extracted from the claims data that are analyzed by the predictive engine 84 include: history of preterm delivery, history of preterm labor, history of hypertension, history of diabetes, current PIH, current gestational diabetes, urinary tract infection, asymptomatic B acteriuria, sexually transmitted infections, HIV infections, history of abortion, number of prenatal visits to date, number of outpatient visits to date, date of last outpatient visit, number of ED visits to date, date of last ED visit, number of inpatient admissions to date, and date of last inpatient admission. In this manner, the system and method 10 is able to stratify, in real-time, the risk of each pregnant patient. Therefore, those patients at the highest risk are automatically identified so that targeted intervention and care may be instituted. Additionally, those patients receiving focused care and intervention may also receive educational materials and reminders so that they may modify any risk behavior that may be harmful to the pregnancy.

One output from the risk computation logic module 80 includes the risk scores of all the pregnant patients for preterm birth. In addition, the module 80 may rank the patients according to the risk scores and provide the identities of those patients at the top of the list. For example, a healthcare provider may desire to identify the top 10% patients most at risk for preterm birth.

Optionally, the risk computation logic module 80 includes an artificial intelligence and/or other fine-tuning techniques and methods 86 that may be used to further refine the predictive engine variables, weights, and thresholds to increase the accuracy of the risk computation over time and perhaps take into account trends and other factors. The artificial intelligence model tuning process 86 may utilize adaptive self-learning capabilities using machine learning technologies. The capacity for automatic self-reconfiguration enables the system and method 10 to be sufficiently flexible and adaptable to detect and incorporate trends or differences in the underlying patient population that may affect the predictive accuracy of a given algorithm. The artificial intelligence model tuning process 86 may periodically retrain the predictive model algorithm for improved accurate outcome to allow for selection of the most accurate statistical methodology, variable count, variable selection, interaction terms, weights, and intercept. The artificial intelligence model tuning process 86 may automatically modify or improve a predictive model in three exemplary ways. First, it may automatically adjust the predictive weights of one or more variables without human supervision. Second, it may automatically adjust the threshold values of specific variables without human supervision. Third, the artificial intelligence model tuning process 86 may, automatically and without human supervision, evaluate new variables present in the data feed but not currently used in the predictive model, which may result in improved accuracy. The artificial intelligence model tuning process 86 may compare the actual observed outcome of the event to the predicted outcome then separately analyze the variables within the model that contributed to the incorrect outcome. It may then re-weigh the variables that contributed to this incorrect outcome, so that in the next reiteration those variables are less likely to contribute to a false prediction. In this manner, the artificial intelligence model tuning process 86 is adapted to reconfigure or adjust the predictive model based on the specific clinical setting or population in which it is applied without manual reconfiguration or modification of the predictive model. The artificial intelligence model tuning process 86 may also be useful to automatically scale the predictive model to different health systems, populations, and geographical areas in a rapid timeframe.

The results from the risk computation logic module 80 are provided to the hospital and/or authorized healthcare personnel, such as the health plan, the intervention coordination team, primary care physician and nurse, OBGYN physician and nurse, and other caretakers by a data presentation logic module 90. The data presentation logic module 90 includes an information and reporting interface 92 that is adapted to provide and present information on the performance of and output from the lightweight clinical pregnancy preterm birth predictive system and method 10. A user (e.g., hospital personnel, administrator, medical personnel, and other intervention coordination team) is able to find specific data they seek through simple and clear visual navigation cues, menus, icons, windows, and other devices. The interface may further be responsive to audible commands, for example. Because the number of patients can be overwhelming, a simple graphical interface that maximizes efficiency and reduces user navigation time is desirable. The visual cues are preferably presented in the context of the problem being evaluated to help pinpoint the issues and factors that are primary contributors to preterm birth. Authorized users may also generate reports that present the information in an easy to comprehend manner, incorporating lists, tables, charts, and other graphics where desirable.

The information and reporting interface 92 allows interactive requesting of a variety of views, reports and presentations of extracted data and risk score calculations from an operation database within the system, including for example, summary views of a list of patient in a specific care location; detailed explanation of the components of the various sub-scores; graphical representations of the data for a patient or population over time; comparison of incidence rates of preterm birth to the rates of prediction in a specified time frame; comparison between healthcare providers, summary text clippings, and other information.

The data presentation logic module 90 further includes a messaging interface 94 that is adapted to generate output messages in formats such as HL7 messaging, text messaging, e-mail messaging, multimedia messaging, web pages, web portals, REST, XML, computer generated speech, constructed document forms containing graphical, numeric, and text summary of the risk assessment, reminders, and recommended actions. The interventions generated or recommended by the system and method 10 through the data presentation logic module 90 may include: risk score report to the primary physician to highlight risk of preterm birth for their patients; risk score report for use by population surveillance of pregnant women identified as having the highest risk; and comparison of aggregate risk of preterm births for a single provider or among many providers to allow risk-standardized comparisons.

The output data from the data presentation logic module 90 may be transmitted wirelessly or via LAN, WAN, the Internet, and delivered to healthcare facilities' electronic medical record stores, user electronic devices (e.g., pager, text messaging program, mobile telephone, tablet computer, mobile computer, laptop computer, desktop computer, and server), and other data stores, databases, devices, and users. The system and method 10 may automatically generate, transmit, and present information such as high-risk patient lists with risk scores, natural language generated text messages, reports, recommended actions, alerts, flags, appointment reminders, and surveys.

The data presentation logic module 90 further includes a patient communication and educational interface module 96. Patients (and appointed family members) may communicate with system 10 via a secure communication channel using a downloaded mobile app and/or text messages. Using this secure communication channel, system 10 may send doctor appointment attendance reminders, appointment scheduling reminders, medication reminders, prescription refill reminders, self-monitoring (e.g., blood pressure) reminders, etc. Further, system 10 may solicit questions to validated survey questions from the patient intended to obtain more detailed and specific information about the patient's health, symptoms, and/or level of satisfaction with the care she has received. System 10 may use advanced analytics and artificial intelligence to tailor the message content, educational content, timing, and frequency to meet patient-specific risk factors and needs. The educational content may include text messages, audio messages, short or comprehensive videos, and URL links to web resources (e.g., websites, documents, articles, videos).

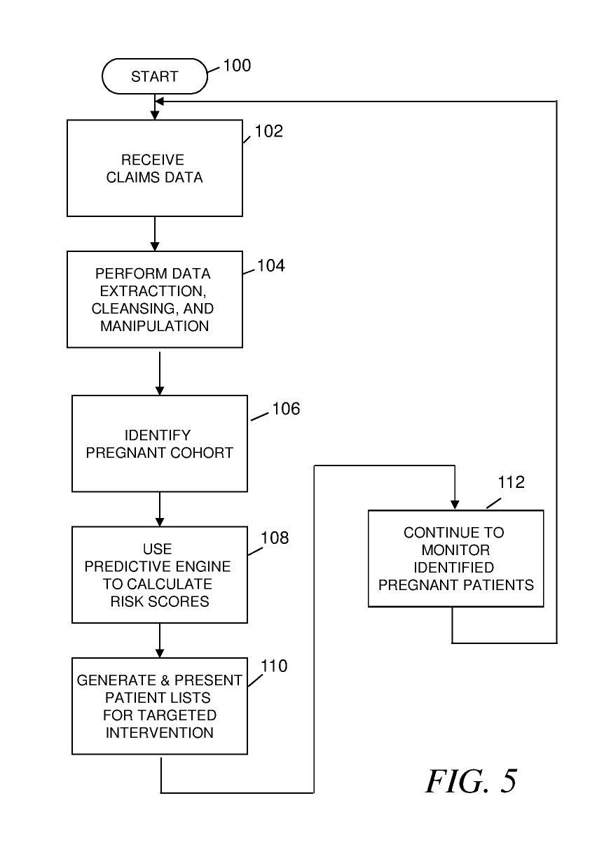

FIG. 5 is a simplified flowchart of an exemplary embodiment of a lightweight clinical pregnancy preterm birth predictive system and method 10 according to the teachings of the present disclosure. The method 100 receives insurance claims data related to specific patients from one or more sources, as shown in block 102. These data may be encrypted or protected using data security methods now known or later developed. In block 104, the method 100 pre-processes the received data, such as data extraction, data cleansing, and data manipulation, as shown in block 104. Other data processing techniques now known and later developed may be utilized. In block 106, pregnant women among the patient population represented by received claims data are identified. For example, pregnant women are those patients whose claims data indicate the patient is a Medicaid Type Programs 40 (TP 40), the patient is receiving prenatal care services; and the patient is receiving pregnancy-related diagnosis or procedure. In block 108, by analyzing the pre-processed data, the predictive engine calculates a risk score for each pregnant patient. In block 110, the method 100 generates one or more lists showing those patients with the highest risk for preterm birth. The results from the predictive engine may be provided, transmitted, and otherwise presented to hospital personnel, such as members of an intervention coordination team and other relevant stakeholders. These lists may be generated on a daily basis or according to another desired schedule. The intervention coordination team may then prescribe and follow targeted intervention and treatment plans for patient care. In block 112, those patients identified as high-risk are continually monitored while they are undergoing more targeted intervention. The method 100 then proceeds to block 102 to process new claims data, if any.

The lightweight clinical pregnancy preterm birth predictive system and method 10 are operable to display, transmit, and otherwise present the list(s) of high risk patients to the intervention coordination team, which may include physicians, physician assistants, case managers, nurses, social workers, family members, and other personnel or individuals involved with the patient's care. The means of presentation may include e-mail, text messages, multimedia messages, voice messages, web pages, facsimile, audible or visual alerts, etc. delivered by a number of suitable electronic or portable computing devices. The intervention coordination team may then prioritize intervention for the highest risk patients and provide targeted inpatient care and treatment. The lightweight clinical pregnancy preterm birth predictive system and method 10 may further automatically present a plan to include recommended intervention and treatment options.

Although the focus of the present disclosure is the analysis of only the patients' health insurance claims data, the data received and analyzed by the system and method 10 may, in alternate embodiments, further include clinical data such as electronic medical records (EMR) and/or electronic health records (EHR). The EMR/EHR clinical data may be received from entities such as hospitals, clinics, pharmacies, laboratories, and health information exchanges, including: vital signs and other physiological data; data associated with comprehensive or focused history and physical exams by a physician, nurse, or allied health professional; medical history; prior allergy and adverse medical reactions; family medical history; prior preterm birth/labor history; prior surgical history; emergency room records; medication administration records; culture results; dictated clinical notes and records; gynecological and obstetric history; mental status examination; vaccination records; radiological imaging exams; invasive visualization procedures; psychiatric treatment history; prior histological specimens; laboratory data; genetic information; physician's notes; networked devices and monitors (such as blood pressure devices and glucose meters); pharmaceutical and supplement intake information; and focused genotype testing. Additional sources or devices of EMR data may provide, for example, lab results, medication assignments and changes, EKG results, radiology notes, daily weight readings, and daily blood sugar testing results. These data sources may be from different areas of the hospital, clinics, patient care facilities, patient home monitoring devices, among other available clinical or healthcare sources.

In an alternate embodiment, the system and method 10 may additionally receive and analyze non-clinical data, which may include, for example, social, behavioral, lifestyle, and economic data; type and nature of employment; job history; medical insurance information; hospital utilization patterns; exercise information; addictive substance use; occupational chemical exposure; frequency of physician or health system contact; location and frequency of habitation changes; predictive screening health questionnaires such as the patient health questionnaire (PHQ); personality tests; census and demographic data; neighborhood environments; diet; gender; marital status; education; proximity and number of family or care-giving assistants; address; housing status; social media data; educational level; marital status; education; community and religious organizational involvement; proximity and number of family or care-giving assistants; census tract location and census reported socioeconomic data for the tract; housing status; number of housing address changes; frequency of housing address changes; requirements for governmental living assistance; ability to make and keep medical appointments; independence on activities of daily living; hours of seeking medical assistance; location of seeking medical services; sensory impairments; cognitive impairments; mobility impairments; educational level; employment; and economic status in absolute and relative terms to the local and national distributions of income; climate data; and health registries. Such data sources may provide further insightful information about patient lifestyle, such as the number of family members, relationship status, individuals who might help care for a patient, and health and lifestyle preferences that could influence health outcomes. The non-clinical patient data may also include data entered by the patients, such as data entered or uploaded to a social media website such as status updates and photographs.

The system and method 10 may be implemented in hardware, software, or a combination of hardware and software. The system and method 10 described herein are configured to harness, simplify, and sort patient information in real-time, predict and identify highest risk patients, coordinate and alert practitioners, and monitor patient outcomes across time and space. The present system and method identifies those patients most in need of intervention to prevent preterm birth, thus leading to better patient outcomes.

The features of the present invention which are believed to be novel are set forth below with particularity in the appended claims. However, modifications, variations, and changes to the exemplary embodiments described above will be apparent to those skilled in the art, and the system and method described herein thus encompasses such modifications, variations, and changes and are not limited to the specific embodiments described herein.

What is claimed is:

1. A lightweight clinical pregnancy preterm birth predictive system, comprising:
a data store configured to receive and store patient data consisting of only health insurance claim data associated with a plurality of patients;
a predictive model including a plurality of weighted risk variables and risk thresholds;
a risk logic module configured to identify a pool of pregnant patients and to apply the predictive model to the patient data of the pool of pregnant patients to determine a risk score for each pregnant patient to identify at least one patient who is at risk for preterm birth; and
a data presentation module operable to present notification and information to an intervention coordination team about the identified at least one high-risk patient.

2. The system of claim 1, further comprising an artificial intelligence tuning module adapted to automatically adjust the weights of the plurality of risk variables in response to trends in the patient data.

3. The system of claim 1, further comprising an artificial intelligence tuning module adapted to automatically adjust the risk thresholds of the plurality of risk variables in response to trends in the patient data.

4. The system of claim 1, further comprising an artificial intelligence tuning module adapted to automatically add or remove risk variables in the at least one predictive model in response to trends in the patient data.

5. The system of claim 1, further comprising an artificial intelligence tuning module adapted to automatically adjust at least one of the weights, risk thresholds, and risk variables in response to trends in the patient data.

6. The system of claim 1, further comprising an artificial intelligence tuning module adapted to automatically adjust a parameter in the predictive model in response to detecting a change in the patient data to improve the accuracy of risk score determination.

7. The system of claim 1, wherein the data store is configured to receive and store real-time and historic patient data.

8. The system of claim 1, wherein the data presentation module is further configured to generate and transmit notification and information to at least one of patient, family, and care team members in a form selected from at least one member of the group consisting of text message, multimedia message, instant message, voice message, e-mail message, web page, web-based message, web pages, web-based message, and text files.

9. The system of claim 8, further comprising an artificial intelligence tuning module adapted to automatically determine content of notification and information generated and transmitted in response to a risk score of at least one patient via the data presentation module.

10. The system of claim 1, wherein the data presentation module is further configured to generate and transmit notification and information to at least one mobile device.

11. The system of claim 1, wherein the data presentation module further comprises a dashboard interface adapted to present and display information in response to a user request.

12. The system of claim 1, wherein the data store is configured to receive and store patient data comprising data related to the patient's medical claims, eligibility for coverage, insurance membership, and healthcare provider.

13. The system of claim 1, wherein the data store is configured to receive and store patient data comprising a social determinant of health.

14. The system of claim 1, wherein the data store is configured to receive and store patient data comprising medical claim data that comprise data on or related to type of claim (inpatient, long term care, prescription drug, etc.), type of service, beginning and end date of service, place of service, type of service, procedure code(s), diagnosis code(s), provider ID(s), patient status, prescribing physician ID, prescription, and prescription fill date.

15. The system of claim 1, wherein the data store is configured to receive and store patient data comprising eligibility data that comprise data on or related to identification number(s), date of birth, gender, race/ethnicity, county, zip code, plan type, basis of eligibility, eligibility group, days of eligibility, and income level.

16. The system of claim 1, further comprising a data integration logic module configured to receive the patient data and perform data extraction and data scrubbing on the received patient data.

17. A lightweight clinical pregnancy preterm birth predictive system, comprising:
a data store configured to receive and store patient data consisting of only health insurance claim data associated with a plurality of patients;
at least one predictive model including a plurality of weighted risk variables and risk thresholds;
a risk logic module configured to identify a pool of pregnant patients and to apply the predictive model to the patient data of the pool of pregnant patients to determine a risk score for each pregnant patient to identify those patients who are at risk for preterm birth;
a data presentation module operable to present notification and information to at least one healthcare provider about the identified at least one high-risk patient; and
an artificial intelligence tuning module configured to automatically adjust at least one of the weighted risk variables and risk thresholds in the predictive model in response to comparing actual patient outcomes and the determine risk scores for the patients.

18. The system of claim 17, wherein the data store is configured to receive and store real-time and historic patient data.

19. The system of claim 17, wherein the data store is configured to receive and store patient data comprising data related to the patient's medical claims, eligibility for coverage, insurance membership, and healthcare provider.

20. The system of claim 17, wherein the artificial intelligence tuning module is further configured to automatically add or remove risk variables in the at least one predictive model in response to trends in patient data.

21. The system of claim 17, wherein the artificial intelligence tuning module is further adapted to automatically adjust at least one of the weights, risk thresholds, and risk variables in response to trends in patient data.

22. The system of claim 17, wherein the data store is configured to receive and store patient data comprising a social determinant of health.

23. The system of claim 17, wherein the data store is configured to receive and store patient data comprising medical claim data that comprise data on or related to type of claim (inpatient, long term care, prescription drug, etc.), type of service, beginning and end date of service, place of service, type of service, procedure code(s), diagnosis code(s), provider ID(s), patient status, prescribing physician ID, prescription, and prescription fill date.

24. The system of claim 17, wherein the data store is configured to receive and store patient data comprising eligibility data that comprise data on or related to identification number(s), date of birth, gender, race/ethnicity, county, zip code, plan type, basis of eligibility, eligibility group, days of eligibility, and income level.

25. The system of claim 17, further comprising a data integration logic module configured to receive the patient data, and perform data extraction and data scrubbing on the received patient data.

26. A lightweight clinical pregnancy preterm birth predictive method, comprising:
receiving and storing patient data consisting of only health insurance claim data associated with a plurality of patients;
identifying at least one pregnant patient from the plurality of patients;
high-risk patient associated with at least one medical condition using at least one predictive model including a plurality of weighted risk variables and risk thresholds in consideration of the clinical and non-clinical data;
applying a predictive model to the patient data to determine a risk score associated with preterm birth for each pregnant patient, and identifying at least one pregnant patient at risk for preterm birth according to the risk score;
presenting a notification to at least one healthcare provider about the identified at least one pregnant patient at risk for preterm birth.

27. The method of claim 26, further comprising automatically monitoring and adjusting parameters in the predictive model in response to trends in the patient data.

28. The method of claim 26, wherein receiving and storing patient data comprises receiving and storing data related to the patient's medical claims, eligibility for coverage, insurance membership, and healthcare provider.

29. The method of claim 26, wherein receiving and storing patient data comprises receiving and storing a social determinant of health.

30. The method of claim 26, wherein receiving and storing patient data comprises receiving and storing data on or related to type of claim (inpatient, long term care, prescription drug, etc.), type of service, beginning and end date of service, place of service, type of service, procedure code(s), diagnosis code(s), provider ID(s), patient status, prescribing physician ID, prescription, and prescription fill date.
